(19) United States
(12) Patent Application Publication
(10) Pub. No.: US 2020/0054544 A1
(43) Pub. Date: Feb. 20, 2020

(54) ELECTROLYTE BALANCED CLEANSING COMPOSITION FOR USE IN THE CLEANING OF MUCOSAL MEMBRANES

(71) Applicant: GPCP IP Holdings LLC, Atlanta, GA (US)

(72) Inventors: Yin Z. Hessefort, Menasha, WI (US); Arinne Lyman, Greenville, WI (US); Daniel Gardner, Tiffin, OH (US)

(21) Appl. No.: 16/537,213

(22) Filed: Aug. 9, 2019

Related U.S. Application Data
(60) Provisional application No. 62/764,846, filed on Aug. 15, 2018.

(51) Int. Cl. A61K 8/81; A61K 8/60; A61K 8/55; A61Q 19/10

(52) U.S. Cl. CPC A61K 8/817; A61K 8/602; A61K 2800/596; A61Q 19/10; A61K 8/55

(57) ABSTRACT

The present disclosure is directed to a cleansing composition and system for cleaning mucosal membranes. The cleansing composition includes an anionic surfactant complex that provides a mild, non-irritating cleanser that when applied to a tissue substrate provides improved sheet properties. The system further includes a dispenser for applying the cleanser to a toilet tissue creating a simple, effective and disposable cleaning system.

### RELATED APPLICATION

This application claims the benefit from Provisional Application No. 62/764,846, which was granted an International filing date of Aug. 15, 2018, which are incorporated herein by reference for all purposes.

This disclosure relates to a cleansing composition and method for cleaning mucosal membranes. More particularly the disclosure relates to a cleansing composition that cleans effectively while being no more irritating than water, i.e., having a (Bovine Corneal Opacity and Permeability Assay) In-Vitro Score (IVIS)—BCOP score—of less than 1.0. Still more particularly, the disclosure relates to a cleansing composition for mucosal membranes that is dilute, e.g., having a solids content of no more than 7%, and comprises a complex of at least one anionic surfactant and at least one non-anionic surfactant. The disclosure further relates to the application of the described composition to a disposable substrate and the synergies that result. Finally, the disclosure relates to a system for applying the cleansing composition to a tissue substrate comprising a dispenser for dispensing the cleanser and a substrate holder for dispensing the tissue substrate.

### BACKGROUND

Products for the cleaning of mucosal membranes have become a staple in home hygiene. Mucosal membranes line all body cavities that open to the exterior of the body including the respiratory, digestive, urinary and reproductive tracts. These membranes are effective barriers, protecting the human body from external pathogens, bacteria, and dirt. The mechanisms that allow mucosal membranes to be effective also require that they be regularly cleaned, which makes them susceptible to irritation, dryness and inflammation.

Constant cleaning of these membranes occurs in both the perineal and perianal regions. Unlike other regions of the body, the perineal and perianal regions are generally protected from environmental grime, like dirt and oil. Typically, these regions are cleaned multiple times daily using dry products such as toilet paper or toilet tissue. However, increasingly, consumers have expressed a desire for the superior cleaning that comes with the use of a cleansing solution.

Manufacturers generally apply cleansing solutions for intimate cleaning to a wiper. Wipers are thick fibrous structures that are generally pre-impregnated with the cleansing solution. Typical intimate wiper products include baby wipes, adult incontinence wipes, feminine care wipes, and the like. For many people, a dry product is completely suitable day to day, but they sometimes want the gentleness and superior cleaning associated with a pre-impregnated wipe. People suffering from pathological conditions, for example, hemorrhoids, irritant bowel syndrome, chronic diarrhea, urine incontinence, menstruation, or fissures, may desire the superior cleaning of a wetted product on a regular basis.

A growing number of consumers are interested in obtaining the superior cleaning associated with wipes, but they also desire a simpler and fully disposable alternative. This has given rise to the development of cleansing solutions and applicator systems that apply those cleansing solutions to common toilet tissue.

Cleansing solutions for use in these systems have to be non-irritating and compatible with the removal of biological waste and fluids. In the art of cleansing, it is well understood that high levels of surfactants and/or other chemicals increases the likelihood of irritation or reaction by the consumer. However, higher levels of surfactants or detergents are generally required to obtain suitable cleaning. The fluid cleansing solutions that are used in wiper products are generally formulated to remain moist over an extended period of storage and therefore, require significantly higher amounts of preservatives, wetting agents and surfactants, besides irritancy, preservatives, for example, are well-known for their sensitizing drawbacks including causing allergies and rashes. By contrast cleansing solutions, like the one described herein, are stored in closed applicators to reduce contamination and do not suffer from the limitations often associated with wiper products. Accordingly, a cleansing solution for use in a toilet tissue applicator system need not be shelf stable once opened to the environment or remain non-evaporative for long period of time as the solution is used immediately upon application. However, the amount of contact between the wetted substrate and the body is very quick so the solution must clean quickly and efficiently.

Other limitations on systems using tissue wetting include difficulty of application of the cleansing solution to the substrate, and the interaction of the cleansing solution with the substrate. If the cleansing solution is difficult to apply or messy, consumers are disappointed. If the solution is easy enough to apply but the coverage amount is too much or too little, or the substrate becomes saturated or falls apart, consumers are again disappointed. For consumers to accept cleansing solutions applied to toilet tissue, the solutions should exhibit good cleaning, be gentle, not leave behind an oily residue, be easy to apply, cover an appropriate area of the tissue substrate, and be disposable and compatible with all types of modern plumbing.

Described herein is a cleansing solution that is specially formulated to be used in a toilet tissue application system for the cleaning of mucosal membranes. The cleaning solution is very dilute, needing only low levels of the cleaning agent because the cleaning agent is effective at cleaning detritus that is predominately water soluble biological waste associated with the perineal and perianal regions. The cleaning agent used in the cleansing solution as described comprises a complex of low concentration of anionic surfactant and a non-anionic, typically cationic, surfactant.

Complexes of anionic surfactants with other, non-ionic, amphoteric and cationic surfactants are well known. As discussed for example, in the background of U.S. Pat. No. 7,157,414, to Johnson and Johnson, when formulating a composition for mildness, anionic surfactants can be associated with amphoteric or cationic surfactants to yield surfactant complexes with significantly less ocular irritation. Johnson and Johnson developed this technology for use in their baby shampoos. In the art of baby shampoo, these cleaning agents are very mild, but they suffer from poor foaming and cleaning. However, in the area of shampoo, the cleaning agent is used on hair which is typically open to the environment and picks up a lot of dirt and grease.

It has been discovered that the surfactant complex, when used in to clean mucosal membranes is both mild and effective. Further when the cleansing composition is combination with a tissue substrate significant benefits occur. The use of the surfactant complex in a composition dispensed onto a substrate provides improvements in the application pattern of the composition. In addition, the cleansing composition including the surfactant complex interacts with the tissue substrate resulting in an improved strength decay timeline keeping the substrate stronger during use without interfering with traditional disposability in modern plumbing.

### SUMMARY OF THE INVENTION

The cleansing solution as described herein provides an appropriate level of clean while being very gentle and non-irritating. The cleansing solution as described provides superior application properties, is completely disposable, and favorably interacts with the tissue substrate, making it a preferred cleanser for use in dry tissue applications. According to one embodiment, the specification describes a cleansing composition for the cleaning of mucosal membrane comprising an aqueous base of at least 93% water, a complex of at least one anionic surfactant and one non-anionic surfactant, wherein the composition has an eye irritancy BCOP score of less than 1.

According to another embodiment, the specification describes a cleansing composition for the cleaning of a mucosal membrane consisting essentially of an aqueous base of at least 93% water and a complex of at least one anionic surfactant and one non-anionic surfactant, wherein the composition has an eye irritancy BCOP score of less than 1.

According to another embodiment, a cleaning system comprising: a tissue substrate; a cleansing composition comprising an aqueous base of at least 93% water and a complex of at least one anionic surfactant and at least one non-anionic surfactant, wherein the composition has an eye irritancy BCOP score of less than 1; and a dispenser for applying the cleansing composition to the tissue substrate.

According to still another embodiment, the specification describes a method of cleaning a mucosal membrane comprising: applying a cleansing composition comprising an aqueous base of at least 93% water, at least one anionic surfactant, at least one cationic surfactant, wherein the composition has an eye irritancy BCOP score of less than 1, to a tissue substrate to moisten the tissue substrate; and contacting the mucosal membrane with the moistened tissue substrate to clean the mucosal membrane.

A better understanding of the various disclosed system and method embodiments can be obtained when the following detailed description is considered in conjunction with the drawings.

### BRIEF DESCRIPTION OF THE DRAWINGS

FIG. 1 illustrates one embodiment of a cleansing solution dispenser and a tissue substrate dispenser usable with the composition and method as described herein.

FIGS. 2A and 2B compare application of a cleansing composition comprising only an anionic surfactant to a toilet tissue substrate with the application of a cleansing product of the instant invention to the same toilet tissue substrate.

### DETAILED DESCRIPTION

The following discussion is directed to various embodiments of the invention. The drawing figures are not necessarily to scale. Certain features of the embodiments may be shown exaggerated in scale or in somewhat schematic form and some details of conventional elements may not be shown in the interest of clarity and conciseness. Although one or more of these embodiments may be preferred, the embodiments disclosed should not be interpreted, or otherwise used, as limiting the scope of the disclosure, including the claims. It is to be fully recognized that the different teachings of the embodiments discussed below may be employed separately or in any suitable combination to produce desired results. In addition, one skilled in the art will understand that the following description has broad application, and the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not structure or function.

As used in the following discussion and in the claims, the terms “including” “is”, “comprising”, “containing”, etc. are used in an open-ended fashion, and thus, should be interpreted to mean “including, but not limited to.” If closed language is included, “consisting,” and “consisting essentially of” it will be given its art recognized meaning.

As used herein “web,” “sheet,” “tissue,” “nascent web,” “tissue product,” “base sheet” or “tissue sheet,” can be used interchangeably to refer to the fibrous web (tissue substrate) during various stages of its development. Nascent web, for example, refers to the embryonic web that is deposited on a forming wire. Once the web achieves about 30% solids content, it is referred to as a tissue or a sheet or a web. Post production, the single-ply of tissue is called a base sheet. The base sheet may be used alone or combined with other base sheets to form a tissue product or a multi-ply product.

The present disclosure relates to a cleansing composition that is formulated for use in cleaning mucosal membranes by application to a dry tissue substrate. The present disclosure further relates to the cleansing system including the cleansing solution, the dispenser, and the disposable tissue substrate. While the cleansing composition is described with regard to the cleaning of mucosal membranes and won't be useful in the cleaning of heavy dirt or oil, it may be used in any environment where gentle cleaning is desired.

The cleansing composition as described comprises a cleaning agent that uses a complex of an anionic surfactant and a non-anionic surfactant. As discussed above, such complexes are available in the prior art. According to one embodiment, the cleaning agent can be any art recognized complex of an anionic surfactant and a non-anionic surfactant in a suitable aqueous cleanser solution that produces a BCOP score of less than one. According to another embodiment, the cleaning agent is a complex of an anionic surfactant and a cationic surfactant in a suitable aqueous cleanser solution that produces a BCOP score of less than one. While the invention will be discussed with respect to the anionic and cationic pairing, the disclosure below will apply equally to other anionic surfactant and non-anionic pairs.

Any art recognized anionic surfactant may be used to form a surfactant complex with a non-anionic surfactant. Anionic surfactants for use in the cleansing composition may include sulfates, sulfonates, phosphate esters, or carboxylates. More particularly, suitable anionic surfactants include alkyl sulfates, alkyl ether sulfates, alkyl monoglyceryl ether sulfates, alkyl sulfonates, alkylaryl sulfonates, sulfonated olefins, alkyl sulfosuccinates, alkyl ether sulfosuccinates, alkyl sulfosuccinamates, alkyl amidosulfosuccinates, alkyl carboxylates, alkyl amidoethercarboxylates, alkyl carbonates, alkyl succinates, fatty acid succinates, fatty acyl sarcosinates, fatty acyl amino acids, fatty acyl taurates, fatty alkyl sulfoacetates, alkyl phosphates, acyl lactylates, protein condensates and mixtures of the same.

Suitable anionic surfactants of the foaming type include, for example, the alkyl sulfates, the alkanesulfonates, the α-olefin sulfonates, the acyl isethionates, the acyl taurides, the acyl sarcosides, the sulfosuccinic acid monoalkyl ester salts and the alkyl polyglycol ether carboxylates in the form of their alkali metal, magnesium, ammonium or alkanolammonium salts.

The anionic surfactants may be present in an amount of from about 0.01% to about 3%, for example, from about 0.05% to about 0.05%, for example, from about 0.1% to about 1.0%.

According to one embodiment, the cleaning agent is a complex of an anionic surfactant and a cationic surfactant. Any art recognized cationic surfactant can be used to create the surfactant complex as described. Cationic surfactants are defined as those surfactants that possess a positive charge and include such surfactant classes as benzalkonium, stearalkonium, Cetrimonium chlorides, trimethyl ammoniums, and methyl sulfates.

Quaternary Ammonium cationic surfactants for use in the cleansing composition may be chosen from one or more primary, secondary or tertiary amines or quaternary ammonium salts. Dodecyl-, Coco-, Hexadecyl-, Octadecyl-, Octadecyl/Behenyl-Behenyl-, cocamidopropyl-, Trimethyl Ammonium Chloride; Coco-, Stearyl-, bis(2-hydroxyethyl) Methyl Ammonium Chloride; Benzalkonium Chloride; Alkyl-, Tetradecyl-, Octadecyl-Dimethyl Benzyl Ammonium Chloride; Dioctyl-, Di(Octyl-Decyl)-, Didecyl-, Dihexadecyl-, Distearyl-, Di(Hydrogenated Tallow)-Dimethyl Ammonium Chloride; Di(Hydrogenated Tallow) Benzyl-, Trioctyl-, Tri(Octyl-Decyl)-, Tridodecyl-, Trihexadecyl-Methyl Ammonium Chloride; Dodecyl Trimethyl-, Dodecyl Dimethyl Benzyl-, Di-(Octyl-Decyl) Dimethyl, Didecyl Dimethyl-Ammonium Bromide.

According to one embodiment, the cationic surfactant is a polymeric cationic surfactant. Cationic polymers suitable for use in the compositions of the present invention include poly ethylene oxide or poly propylene oxide block copolymers, ethoylated or sulfonated resins, carboxymethyl cellulose, polyquaterniums, polysaccharides, polyacrylates, xanthane; copolymers of dimethylaminoethylmethacrylate and acrylamide; copolymers of dimethyldiallylammonium chloride and acrylamide.

Polyquaterniums are well known cationic polymers recognized by the Personal Care Products Counsel (PCPC), as cosmetic raw materials. Polyquaterniums are distinguished by the number following their name. Numbers have been assigned in the order of registration, not based upon chemical structure. Examples of polyquaterniums for use in the instant composition include, but are in no way limited to, polyquaternium-1, polyquaternium-2, polyquaternium-5, polyquaternium-6, polyquaternium-7, polyquaternium-8, polyquaternium-9, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30 polyquaternium-32, polyquaternium-33, polyquaternium-34, polyquaternium-35, polyquaternium-36, polyquaternium-37, polyquaternium-39, polyquaternium-43, polyquaternium-44, polyquaternium-46, polyquaternium-47, polyquaternium-49, polyquaternium-51, polyquaternium-52, polyquaternium-53, polyquaternium-55, polyquaternium-57, polyquaternium-61, polyquaternium-64, polyquaternium-65, and mixtures thereof.

The cationic surfactants may be present in an amount of from about 0.01% to about 3%, for example, from about 0.05% to about 0.05%, for example, from about 0.1% to about 1.0%.

The surfactant complex may be made via any conventional method for combining two or more fluids, for example, pouring, mixing, adding dropwise, pumping, etc. In an aqueous solution, the anionic components and the cationic component will be naturally attracted to one another by their differing electrical charges. The complex is the electrostatic attraction between the two surfactants and may be loose or strong depending upon the particular surfactants used.

According to one embodiment, the surfactant complex is present in the aqueous cleanser solution in an amount of form 0.01% to about 6%, for example, less than 5%, for example, less than 4%, for example, less than 3%, for example, less than 2%, for example from about 0.01% to about 2%.

The cleansing composition as described can further include one or more optional ingredients. Optional ingredients can include one or more additional cleaning agents, as well as preservatives, dyes, moisturizing agents, glycols, skin conditioning agents, thickeners, solvents, vitamins, anti-oxidants, pH modifiers, film formers, anti-inflammatories, colorants, humectants, emollients, fragrances, extracts, including for example, botanical extracts or marine extracts, silicones, emollients, pigments, foam boosting, UV absorbents.

According to one embodiment, other cleaning agents that may be compatible with the surfactant complex described above may be included in the cleansing composition. Compatible cleaning agents may include non-ionic surfactants, hydrotropes, chelating agents, preservatives, alcohols, e.g., ethanol, and biocidally active botanical extracts, for example, essential oils, and like.

These compatible active agents may be present in an amount of from about 0.01% to about 2.0%, for example, from about 0.05% to about 1.0%, for example, from about 0.1% to about 0.5%.

While the cleansing composition as described uses anionic and cationic surfactants for detergency, surfactants may be added to the cleansing composition for other purposes, for example, as film formers or skin conditioning agents, etc. The cleansing compositions as described herein may optionally comprise one or more additional surfactants chosen from amphoteric surfactants, non-ionic, anionic surfactants, cationic surfactants, or non-ionic surfactants. Like the primary anionic and cationic surfactants, any additional surfactants should not disrupt the electrolytically balanced surfactant complex.

Optional amphoteric surfactants for use in the cleansing composition as described, include but are not limited to cocamidopropyl betaine marketed under the tradename AMPHOSOL HCP-HP both from Stepan Co, lauryl betaine. Appropriate amphoteric surfactants are readily available and are marketed by companies such as Akzo Nobel, Pilot and Solvay Chemical. Amphoteric surfactant may be present in the cleansing composition in an amount of from about 0.01% to about 2.0%, for example, from about 0.05% to about 1.0%, for example, from about 0.1% to about 0.5%.

Optional non-ionic surfactants for use in the cleansing composition as described include, but are not limited to alkanol amines, alkanolamides, ethoxylated amides, ethoxylated fatty acids, ethoxylated fatty alcohols, alkoxylated esters, alkyl polyglucosides, alkoxylated triglycerides, sorbitan esters and sorbitan ethers.

These non-ionic surfactants can be present in the cleansing composition in an amount of from about 0% to about 1.0%, for example, from about 0.01% to about 0.5%, for example, from about 0.1% to about 0.5%.

The cleansing composition as described comprises an aqueous base of deionized water. The composition is at least about 93% water, for example at least about 94% water, for example, at least about 95% water, for example, at least about 95.5% water, for example, about 96% water.

According to one embodiment, the aqueous base for the cleansing product may have low levels of petroleum jelly. The petroleum jelly may be emulsified in the water base to form a lotion/water.

Other optional ingredients and excipients that may also be added to the formulation include, for example, emollients, fragrances, dyes, humectants, moisturizing agents, skin conditioning agents, chelating agents, preservatives, keratolytic, thickeners, solvents, botanicals, extracts, vitamins, anti-oxidants, pH modifiers, film formers, anti-inflammatories, abrasives, colorants, and the like.

Depending upon the embodiment, optional stabilizers may be used to inhibit reactions between ingredients and to maintain the homogeneity of the composition. According to one embodiment, if the cleansing composition is a foaming composition, it may include one or more foam boosters or foam stabilizers. Suitable stabilizers can be chosen from alkyl polyglucosides, amphoteric surfactants, non-ionic surfactants, amide oxides. The stabilizer will be present in the cleansing composition in an amount of from about 0% to about 10%, for example from about 0.01% to about 5%, for example, from about 0.01% to about 2%.

Appropriate emulsifier for use in the cleansing composition as described will be readily included in the art of formulation to create additional lubricity between tissue substrates and mucosal membrane. Suitable emulsifiers can be chosen from glyceryl stearate, stearic acid, stearyl alcohol, ceteareth-20, ceteareth-18, polysorbate 20, polysorbate 80, cetyl alcohol, and stearyl alcohol,

Other emulsifiers can be chosen from polyethoxylated fatty acids, ethoxylated esters, unethoxylated sugar esters, polyoxyethylene fatty ether phosphates, fatty acid amides, phospholipids, polypropoxylated fatty ethers, acyl lactylates, polyethoxylated poly (oxypropylene) glycols, polypropoxylated poly (oxyethylene) glycols, poly (oxyethylene) poly(oxypropylene) ethylene diamines, and mixtures thereof. Examples of such emulsifiers include polyoxyethylene stearate, myristyl ethoxy palmitate, methyl glucoside sesquistearate, sucrose distearate, sucrose laurate, sorbitan monolaurate, polyoxyethylene oleyl ether phosphate, polyoxyethylene oleyl ether phosphate, lauric diethanolamide, stearic monoethanolamide, lecithin, lanolin alcohol propoxylates, sodium stearoyl-2-lactylate, and alcium stearoyl-2-lactylate.

Appropriate solubilizers for use in the cleansing compositions as described will be readily apparent to the skilled artisan and can include hydrotropes, non-ionic, surfactants, chelating agents, builders and the like. According to one embodiment, the solubilizer may be chosen from one or more butanediols, butylene glycols, pentylene glycols, and propandiols, for example, 1,3-butanediol or 1,3-propanediol. The solubilizer can be present in the cleansing composition in an amount of from about 0% to about 5%, for example, from about 0% to about 2.0%, for example, from about 0.1% to about 2.0%, for example from about 0.1% to about 1.6%.

Film-formers for use in the cleansing compositions as described can include polymers, including for example, polyquaterniums or polyacrylic acid. The film-forming agent can be present in the cleansing composition in an amount of from about 0% to about 8%, for example, from about 0.1% to about 5%, for example, from about 0.3% to about 3%, for example, from about 0.5% to about 2%.

Generally, emollients lubricate, soothe, and soften the skin surface. Exemplary emollients include silicons, for example, dimethicone, trisiloxane, dimethiconol, amodimethicone, dimethicone polymers, trimethylsiloxysilicate, polymethylsilsesquioxane, polypropylsilsesquioxane; ethoxylated or propoxylated oily or waxy ingredients such as esters, ethers, fatty acids, fatty alcohols, hydrocarbons, lanolin, and the like. The emollients can be present in the cleansing composition in an amount of from about 0% to about 8%, for example, from about 0.1% to about 3%, for example, from about 0.05% to about 1%.

Humectants are hydroscopic agents that are widely used as moisturizers. Their function is to prevent the loss of moisture from the skin and to attract moisture from the environment. Common humectants include, for example, glycols, sodium PCA, glycerin, propylene glycol, butylene glycol, pentylene glycol, betaine, sodium hyaluronate, sorbitol, urea, hydroxyethyl urea, and the like. The humectants can be present in the cleansing composition in an amount of from about 0% to about 5.0%, for example, from about 0.1% to about 2.5%, for example, from about 0.1% to about 1.5%.

Preservatives for increasing the shelf life of the cleansing composition may also be used. Exemplary suitable preservatives include, but are not limited to disodium EDTA; tetrasodium EDTA; iodopropynyl butylcarbamate; benzoic esters (parabens), such as methylparaben, propylparaben, butylparaben, ethylparaben, sodium methylparaben, and sodium propylparaben; sodium benzoate, phenoxyethanol; benzyl alcohol; phenethyl alcohol; imidiazolidinyl urea; diazolidinyl urea; citric acid, lactic acid, kathon, phenoxyethanol, 2-bromo-2 nitro-propane-1, 3-diol, potassium sorbate, and the like. The preservatives can be present in the cleansing composition in an amount of from about 0.1 to about 3%, for example, from about 0.1% to about 1%, for example, from about 0.1% to about 0.7%, for example, about 0.6%.

Suitable skin conditioning agents include, for example, conditioning polymers, hydrolyzed plant proteins such as hydrolyzed wheat protein, hydrolyzed soy protein, hydrolyzed collagen, and the like. The skin conditioning agents can be present in the cleansing composition in an amount of from about 0% to about 10%, for example, from about 0.1% to about 5%, for example, from about 0.1% to about 3%.

The pH of the system is maintained from about 4.0 to about 6.5, for example, from about 4.2 to about 4.8. Art recognized acids and bases may be used to modify the pH for the cleansing product. Some examples of basic pH modifiers that may be used in the cleansing compositions of the present disclosure include, but are not limited to, ammonia; sodium, potassium, and lithium hydroxide; sodium, potassium, and lithium metal silicates; monoethanolamine; triethylamine; isopropanolamine; ethanolamine; and triethanolamine. Acidic pH modifiers that may be used in the formulations of the present disclosure include, but are not limited to, mineral acids; carboxylic acids; and polymeric acids, including by way of example, citric acid. The pH modifiers will be used in an amount necessary to achieve the desired pH. For example, the pH modifiers can be present in the cleansing composition in an amount of from about 0% to about 5%, for example, from about 0.05% to about 3%, for example, from about 0.1% to about 2%.

A chelating agent is a substance whose molecules can form one or more bonds with a metal ion. In particular, water that may be contained in the cleansing composition often contains metal ions, such as calcium ions, that might react with anionic components (e.g., acids) present within the composition. Some examples of chelating agents that may be used in the cleansing composition of the present disclosure include, but are not limited to, ethylenediamines, ethylenediaminetetraacetic acids (EDTA) acids and/or salts thereof, for example, tetrasodium EDTA, citrate, pyrithione, N,N′-bis(o-hydroxybenzyl)ethylenediamine-N,N′diacetic acid; ethylenebis-N, N′-(2-o-hydroxyphenyl)glycine, 1,3-diaminopropane-N,N,N′, N′-tetraacetic acid; ethylenediamine-N,N′-diacetic acid; ethylenediamine-N,N′-dipropionic acid dihydrochloride; ethylenediamine-N, N′-bis(methylenephosphonic acid); N-(2-hydroxyethyl)ethylenediamine-N,N′, N′-triacetic acid; ethylenediamine-N,N,N′,N′-tetrakis(methylenephosphonic acid); O,O′-bis(2-aminoethyl)ethyleneglycol-N,N,N′,N′-tetraacetic acid; N,N-bis(2-hydroxybenzyl)ethylenediamine-N, N-diacetic acid; 1,6-hexamethylenediamine-N, N,N′, N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; nitrilotris(methylenephosphonic acid); and triethylenetetramine-N,N,N′, N″,N′″, N′″-hexaacetic acid, glucuronic acids and/or salts thereof, succinic acid and/or salts thereof, for example, trisodium ethylenediamine disuccinate; polyphosphates, organophosphates, and the like. The chelating agent can be present in the cleansing composition in an amount of from about 0% to about 5%, for example, from about 0.01% to about 3%, for example, from about 0.5% to about 2%.

Fragrances and dyes may be used in the cleansing compositions as appropriate to appeal to the purchasing consumer. Fragrances and dyes can be present in the cleansing composition in an amount of from about 0% to about 3%, for example, from about 0.1% to about 1%, for example, from about 0.2% to about 0.8%. Fragrances and dyes may be combined with one or more solubilizers to aide in incorporation of the fragrance or dye to the cleansing composition.

Colorants may be used in the cleansing composition as desired. Typical colorants include liquid dyes, pigments and oxides all of which are readily available from commercial suppliers.

UV absorbers may be added to the cleansing composition if desired. Typical UV absorbers include anthranilates, octyl methoxycinnamates, octyl salicylates, oxybenzones, benzophenones, dibenzoyl methanes, avobenzone, and the like.

Moisturizing agents for use in the cleansing compositions as described can include, but are not limited to glycol, collagen; lecithins; liposomes; peptides; polysaccharides; glycerin; sorbitol; propylene glycol; calcium pantothenate; urea; caprylyl glycol; butylene glycol; glucose; magnesium lactate; potassium chloride; potassium lactate; ethylhexylglycerin; dipropylene glycol; silicones, such as dimethicone and cyclomethicone; fatty acids, for example, lanolin acid; fatty alcohols, for example, lanolin alcohol; hydrocarbon oils and waxes; petrolatum; polyhydric alcohols; sterols, for example, cholesterol; vegetable and animal fats, for example, cocoa butter, vegetable waxes, carnauba wax, wax esters, and bees wax; hyaluronic acid, ceramics; caprylic/capric triglycerides; magnesium aspartame; potassium aspartame; sarcosine; and the like. The moisturizing agent can be present in the cleansing composition in an amount of from about 0% to about 10%, for example, from about 0.1% to about 5%, for example, from about 0.3% to about 3%, for example, from about 0.5% to about 2%.

Keratolytics are peeling agents that cause softening and shedding of the outer layer of skin. Keratolytic agents for use in the cleansing composition as described, can include compounds based upon urea, for example, allantoin, or salicylic acid. The keratolytic agent can be present in the cleansing composition in an amount of from about 0% to about 10%, for example, from about 0.1% to about 5%, for example, from about 0.1% to about 3%, for example, from about 0.1% to about 2%.

Thickeners for use in the cleansing composition as described include, for example, cetyl alcohol, stearyl alcohol, carnauba wax, and stearic acid, carboxyethyl cellulose, carboxymethyl cellulose, guar gum, xanthan gum, gelatin, silica, bentonite, silicates, carbomer polymers, and the like. Thickeners can be present in the cleansing composition in an amount of from about 0% to about 5%, for example, from about 0.1% to about 3%, for example, from about 0.2% to about 1%.

Botanicals for use in the cleansing compositions as described may include, for example, aloe vera, green tea extract, cucumber extract, chamomile, oat, Aspen Bark, Bamboo Leaf, Banaba Leaf, Burdock Root, Chamomile, Chrysanthemum, Cucumber Peel, Ginkgo Biloba Leaf, Ginseng Root, Grape Seed, Green Tea, Honey Suckle Flower, Horse Chest Nut, Licorice Root, Maca, Milk Thistle (Silymarin), Olive Leaf, Rosehips, Rosemary, Sacha Inchi, Sea Buckthom, Sunflower, Thyme, White Willow Bark, and the like. Botanicals can be present in the cleansing composition in an amount of from about 0% to about 5%, for example, from about 0.1% to about 3%, for example, from about 0.1% to about 1%.

Vitamins for use in the cleansing composition may include for example, Vitamins A, B, C, D, E, tocopheryl acetate, retinyl palmitate, panthenol, and ascorbic acid. Vitamins can be present in the cleansing composition in an amount of from about 0% to about 5%, for example, from about 0.1% to about 3%, for example, from about 0.1% to about 1%.

Antioxidants for use in the cleansing composition as described can include one or more of glutathione, superoxide dismutase, ubiquinone, omega-fatty acids, Vitamin C, Beta-Glucan, Thioctic Acid, Magnesium Ascorbyl, Phosphate, Ferulic Acid, Superoxide Dismutase, Epigallocatechin Gallate, Ergothioneine, Glutathione, Xanthophylls, and the like. Antioxidants may be present in the composition in an amount of from about 0% to about 5%, for example, from about 0.1% to about 3%, for example, from about 0.1% to about 1%.

The artisan skilled in the formulation of cleansers understands that ingredients may be selected to provide more than one function in a composition. Thus, a single ingredient may be chosen to act, for example, as a pH modifier and a preservative, or as a moisturizer and as a humectant.

According to one embodiment, the cleansing composition as described includes at least one cleaning agent comprising a complex of an anionic surfactant and a cationic surfactant, at least one solubilizer, at least one chelating agent, at least one skin conditioner, at least one film-former, at least one moisturizer or emollient, at least one keratolytic, at least one botanical, and at least one preservative. According to this embodiment, the cleansing composition has an aqueous base having at least about 93% water.

According to another embodiment, the cleansing composition as described includes at least one cleaning agent comprising a complex of an anionic surfactant and a cationic surfactant at least one solubilizer, at least one chelating agent, and at least one skin conditioner.

According to another embodiment, the cleansing composition as described includes at least one cleaning agent comprising a complex of an anionic surfactant and a cationic surfactant, at least one solubilizer, at least one preservative, and at least one skin conditioner.

According to another embodiment, the cleansing composition as described includes at least one cleaning agent comprising a complex of an anionic surfactant and a cationic surfactant, at least one skin conditioner, and at least one preservative. According to still another embodiment, the cleansing composition as described includes at least one cleaning agent comprising a complex of an anionic surfactant and a cationic surfactant, at least one chelating agent, and at least one preservative.

The cleansing composition as described is preferably applied to a tissue substrate by an applicator which forms a part of a dispenser or dispensing system. According to one embodiment, the dispenser is a touchless dispenser that is motion activated. According to this embodiment, the cannister carrying the cleansing solution never comes into contact with the substrate or the user's hand keeping the system closed and preventing the introduction of contaminants or pathogens to the cleansing solution. Because the cleansing solution is protected from the environment, the formulation may comprise fewer and/or less intense preservatives which are known to be irritating, for example, causing allergies or rashes when applied to human skin.

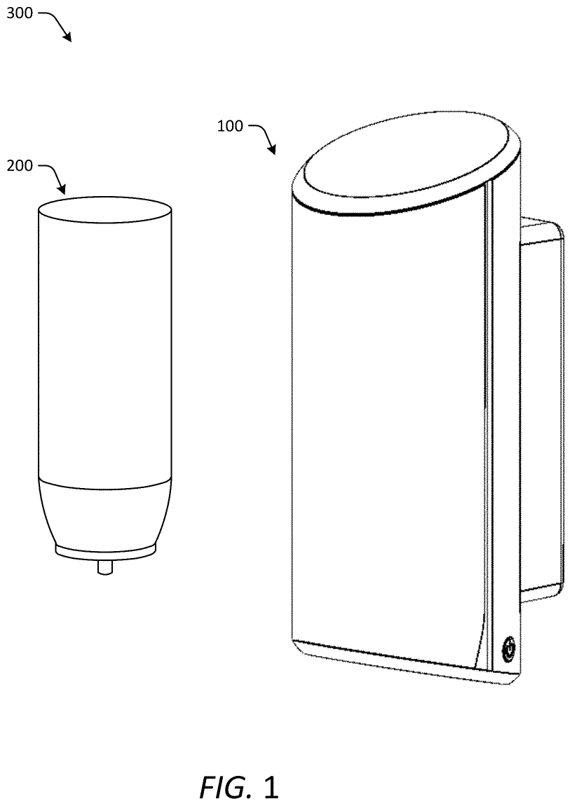

FIG. 1 illustrates one embodiment of a dispenser system 300 including a dispenser 100 and a canister 200 for carrying a cleansing solution as described. Various types of product dispensers 100 are known in the art, including mechanical and automated dispensers configured to dispense a cleansing composition from a supply of the product supported by the dispenser. The supply of cleanser is generally provided in a container 200, for storing the cleanser prior to dispensing from the dispenser 100. The container 200 may be refilled upon depletion of the supply of cleanser, or the container 200 may be replaced with a new prefilled container upon depletion of the supply of cleanser in the original container 200. Soap or cleanser dispensers are generally configured to dispense cleanser in a downward direction onto a user's hand or onto a substrate, such as a sheet product, held by the user's hand. Alternatively, the product may be dispensed by spraying at any preferred angle. Any art recognized applicator 200, dispenser 100 or dispensing system 300 may be used with the cleansing composition as described. According to a preferred embodiment, the dispenser is a touchless dispenser.

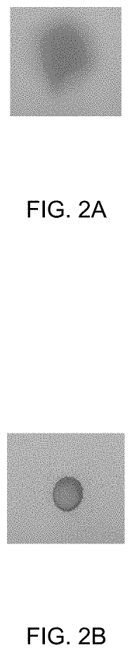

As seen in FIG. 2A when surfactant solution is sprayed on a substrate surface, the spray pattern is spread out and varies in spray angles. However, as seen in FIG. 2B, when the cleansing composition with cationic polymer as described is applied to the substrate, the composition results in a spray pattern that is tightly centered on the tissue substrate. Without wishing to be bound by theory, it is believed that the electrical neutrality of the composition as described lessens the interactions between the composition and the dispenser and the composition and the surrounding atmosphere, creating less interference. In addition, the electrostatic attraction between the anionic surfactant and a polymeric cationic surfactant, cause the spray to be held more closely together resulting in a tighter more controlled spray pattern.

The cleansing composition can be applied to a toilet tissue product that is disposable under ordinary use conditions. The tissue product for use in the system as described, can include any tissue product that is available on the market.

Typical tissue products are made from cellulosic fibers, commonly referred to as wood fibers. Specifically, the base sheet for a tissue substrate for use with the cleansing composition as described can be produced from hardwood (angiosperms or deciduous trees) or softwood (gymnosperms or coniferous trees) fibers, and any combination thereof. Hardwood fibers include, but are not limited to maple, birch, aspen and eucalyptus. Hardwood fibers generally have a fiber length of about 2.0 mm or less. Softwood fiber includes spruce and pine, and exhibit an average fiber length of about 2.5 mm. Cellulosic fibers from diverse material origins may also be used to form the substrate of the present disclosure. The tissue substrate of the present disclosure may also include recycled or secondary fiber. The products of the present disclosure can also include synthetic fibers as desired.

Papermaking fibers for use in the substrates as described can be liberated from their source material by any one of the number of chemical pulping processes familiar to one experienced in the art including sulfate, sulfite, polysulfite, soda pulping, etc. The pulp can be bleached as desired by chemical means including the use of chlorine, chlorine dioxide, oxygen, etc. Alternatively, the papermaking fibers can be liberated from source material by any one of a number of mechanical/chemical pulping processes familiar to anyone experienced in the art including mechanical pulping, thermomechanical pulping, and chemithermomechanical pulping. These mechanical pulps can be bleached, if one wishes, by a number of familiar bleaching schemes including alkaline peroxide and ozone bleaching.

In the production of tissue substrates for use in the instant system, the fiber is generally fed into a headbox where it will be admixed with water and chemical additives, as appropriate, before being deposited on the forming wire. The chemical additives for use in the formation of the base sheets can be any known combination of papermaking chemicals. Such chemistry is readily understood by the skilled artisan and its selection will depend upon the type of end product that one is making. Papermaking chemicals include, for example, strength agents, softeners and debonders, creping modifiers, sizing agents, optical brightening agents, retention agents, and the like.

Typically, a first nascent web is formed from the pulp. The web can be formed using any of the standard equipment known to the skilled artisan, e.g., crescent former, suction breast roll, twin-wire former, etc. The web is transferred from the forming wire to a fabric for non-compactive, e.g., vacuum suction, or limited compactive dewatering. Thereafter, the partially dewatered web is dried without compression by passing hot air through the web while it is supported by the fabric.

The web is then calendered and rolled to await converting. Converting refers to the process that changes or converts base sheets into final products. Typical converting in the area of toilet or facial tissue includes embossing, perforating, and plying.

According to one embodiment, when the cleansing composition as described herein is applied to the substrate, the cleanser delays the decay of the wet strength of the product thereby effectively increasing the temporary wet strength of the tissue substrate. Wet strength agents are typically used in toilet tissue to keep the substrate strong enough to achieve it's intended purpose without ripping, tearing or breaking. Both permanent and temporary wet strength agents are generally included. Temporary wet strength agents are important because in toilet tissue products, the tissue needs to be strong enough to be effective during use, but also need to lose its strength and be disposable. Temporary wet strength agents break down in water, allowing water to penetrate through and come into direct contact with cellulosic substrate causing the substrate to break down and be flushable with standard plumbing.

Because the cleansing composition as described increases the temporary wet strength of the tissue upon application, the tissue may be produced with lower levels of chemicals and still achieve the desired wet strength. Lowering the amount or eliminating the need for wet strength agents during tissue production improves both the cost and environmental impact of the tissue product.

In practice, a tissue substrate of desired length is separated from a tissue roll and held under a dispenser, such as the one shown in FIG. 1. The dispenser is actuated and an appropriate amount of cleansing solution is delivered to the tissue. The moistened tissue is then used to clean the mucosal membrane and surrounding area. Given the brevity of contact between the substrate and the mucosal membrane, the cleansing composition as described has to have sufficient cleansing power to be reasonably fast acting.

According to one embodiment, the cartridge or cannister holding the cleansing solution is loaded to a touchless dispenser. A piece of substrate tissue is removed and held under the motion sensor. The dispenser senses the presence of the tissue substrate and releases an amount of cleansing solution to the surface of the substrate. The dispenser returns to the closed position and the tissue is used to clean one or more areas. According to this embodiment, the dispenser and cleansing solution are not in contact with human skin, making the solution more stable without the need for many preservatives. This closed system is one reason that the irritating surfactants and preservatives can be reduced. This barrier between the cleansing solution and any contact with skin or paper results in a cleansing composition that can be at least 93% water with very low preservative levels, less than 2%.

According to one embodiment, the tissue substrate is packaged along with the cleansing solution as described. According to another embodiment, the tissue and cleansing compositions are packaged separately.

The following examples provide representative embodiments. The methods and products described herein should not be limited to the examples provided. Rather, the examples are only representative in nature.

### EXAMPLES

### Example 1

A cleansing composition as described with the ingredients as set forth in Table 1 was produced and compared to a similar composition using only the cationic surfactant.

TABLE 1

| | Base Formula |
|---|---|
| Ingredient | (Wt. %) |
| Water | 95.5 |
| Anionic surfactant: | 0.4 |
| Sodium Lauryl Glucose | |
| Carboxylate (and) Lauryl | |
| Glucoside | |
| Cationic surfactants: | 0.65 |
| Cocamidopropyl PG- | |
| Dimonium chloride | |
| phosphate; and | |
| Polyquaternium-39 | |
| Chelating Agents | 0.1 |
| Moisturizer and emollients | 0.65 |
| Solvents | 1.6 |
| Fragrance System | 0.5 |
| Preservatives | 0.6 |

The two formulations were subjected to both skin and eye irritation tests. The skin irritation test used was the EpiDerm MTT ET-50 test developed by MatTek. The test consisted of a topical exposure of the cleansing composition to a reconstructed human epidermis (RHE) model followed by a cell viability test. Cell viability was measured by dehydrogenase conversion of MTT [(3-4,5-dimethyl thiazole 2-yl) 2,5-diphenyltetrazoliumbromide], present in cell mitochondria, into a blue formazan salt that was quantitatively measured after extraction from the tissues. The reduction of the viability of tissues exposed to the cleansing composition in comparison to negative controls (treated with water) was used to predict the skin irritation potential. The test scoring was as follows:

EpiDerm MTT ET-50

| Score | | |
|---|---|---|
| >24 | hrs | Non-irritant |
| 12-24 | hrs | Very mild |
| 4-12 | hrs | Moderate to mild |
| 0.5-4 | hrs | Moderate irritant |

The eye irritation test was the Bovine Cornea Opacity/Permeability test, known as BCOP. The BCOP is an in vitro test method that can be used to classify substances as ocular corrosives and severe irritants'. The BCOP uses isolated corneas from the eyes of cattle slaughtered for commercial purposes, thus avoiding the use of laboratory animals. Each treatment group (test substance, negative/positive controls) consists of a minimum of three eyes where the cornea has been excised and mounted to a holder. The cleansing composition was applied to adequately cover the epithelial surface. Toxic effects to the cornea were measured as opacity and permeability, which when combined gave an In Vitro Irritancy Score (IVIS) for each treatment group. A substance that induces an IVIS superior or equal to 55.1 is defined as a severe irritant. The test scoring was as follows:

Eye Irritation (BCOP)

| Score | |
|---|---|
| <0 | Non-Irritant |
| 0-25 | Mild Irritant |
| 25.1-55 | Moderate Irritant |
| >=55 | Severe Irritant |

The results of the testing are set forth in Table 2, below.

TABLE 2
Irritation Test Results

| | | | Eye Irritation | |
|---|---|---|---|---|
| | Skin Irritation Test | | Test (BCOP) | |
| | Test Score | Test | Test | |
| Article | EpiDerm MTT ET-50 | Results | Score | Test Results |
| Name | (hrs) | Rank | BCOP | Rank |
| Pure cationic | 7.6 | Moderate | 28.2 | Moderate |
| surfactant | | to mild | | irritant |
| cleansing | | | | |
| formulation | | | | |
| Elelctrolyte | 25 | Non- | −0.5 | Non-irritant |
| balanced | | irritant | | |
| cleansing | | | | |
| formulation | | | | |

### Example 2

A cleansing composition comprising only an anionic surfactant was compared with the same composition which was modified by the addition of a cationic surfactant to result in a complexing of the anionic surfactant with the cationic surfactant. As can be seen in FIGS. 2A and 2B, the cleansing composition having the complexed surfactant as described herein (seen in FIG. 2B) provided a superior application pattern when compared to that of the anionic surfactant alone (seen in FIG. 2A).

### Example 3

The cleansing composition of Example 1 was applied to two different tissue substrates, the first being a premium tissue product and the second being a non-premium tissue product. Premium tissue products are characterized by the use of better fiber and more expensive processing resulting in a product having higher strength, higher caliper and better softness. The results are set forth in Tables 3 and 4, below.

TABLE 3
Temporary Wet Tensile Strength on Non-premium Tissue

| Non-premium Tissue Substrate | |
|---|---|
| Test 1 | Test 2 |

| | Wet Tensile | | Wet Tensile | |
|---|---|---|---|---|
| Sample | Finch (CD) | | Finch (CD) | |
| Name | Avg | Stdv | Avg | Stdv |
| Water | 30.76 | 1.09 | 28.97 | 0.14 |
| Comp | 46.16 | 1.99 | 42.01 | 2.29 |
| of Ex. 1 | | | | |

TABLE 4
Temporary Wet Tensile Strength on Premium Product

| | Premium Product | | | | | |
|---|---|---|---|---|---|---|
| | Test 1 | | Test 2 | | Test 3 | |
| | Wet Tensile | | Wet Tensile | | Wet Tensile | |
| Sample Name | Finch (CD) Avg | Stdv | Finch (CD)Avg | Stdv | Finch (CD) Avg | Stdv |
| Water | 73.81 | 4.71 | 72.69 | 0.07 | 73.81 | 4.71 |
| Comp. of Ex. 1 | 85.68 | 1.04 | 77.28 | 5.41 | 85.68 | 1.04 |

Wet Tensile—

The wet tensile of the tissue substrate was measured generally following Technical Association of the Pulp and Paper Industry (TAPPI) Method T 576 pm 7, using a three-inch (76.2 mm) wide strip of tissue that is folded into a loop, clamped in a special fixture termed a Finch Cup, then immersed in water. A suitable Finch cup, 3-in., with base to fit a 3-in. grip, is available from:

High-Tech Manufacturing Services, Inc.
3105-B NE 65^{th} Street
Vancouver, Wash. 98663
360-696-1611
360-696-9887 (FAX).

For fresh base sheet and finished product (aged 30 days or less for towel product, aged 24 hours or less for tissue products containing wet strength additive), the test specimens are placed in a forced air oven heated to 105° C. (221° F.) for five minutes. No oven aging is needed for other samples. The Finch cup is mounted onto a tensile tester equipped with a 2.0 pound load cell with the flange of the Finch cup clamped by the tester's lower jaw and the ends of tissue loop clamped into the upper jaw of the tensile tester. The sample is immersed in water that has been adjusted to a pH of 7.0±0.1 and the tensile is tested after a 5 second immersion time using a crosshead speed of 2 inches/minute. The results are expressed in g/3 in., dividing the readout by two to account for the loop as appropriate.

Normally when a tissue contacts water, the cellulose bonds are quickly degraded. To prevent this from happening, the tissue is often treated with either temporary wet strength agents or permanent wet strength agents. Here, for a toilet tissue product, the system would include only temporary wet strength agents. Upon contact with water, the effectiveness of the temporary wet strength agents begins to decay and the tissue substrate loses strength. As can be seen from Tables 3 and 4, when the cleansing composition as described is applied to the tissue product, the strength reduction is significantly less than when the same substrate is moistened with water. The improved strength retention means that the tissue substrate will have more strength for the consumer during use, or that the chemical load of strength agents can be reduced, either of which is beneficial.

Other embodiments of the present invention can include alternative variations. These and other variations and modifications will become apparent to those skilled in the art once the above disclosure is fully appreciated. It is intended that the following claims be interpreted to embrace all such variations and modifications.

What is claimed is:

1. A cleansing composition for the cleaning of skin or a mucosal membranes comprising:
at least one cleaning agent comprising a complex of an anionic surfactant and a non-anionic surfactant;
at least about 93% water; and
wherein the composition has an eye irritancy BCOP score of less than 1.

2. The cleansing composition of claim 1, wherein the non-anionic surfactant is a cationic surfactant.

3. The cleansing composition of claim 2, wherein the cationic surfactant is a polymeric cationic surfactant.

4. The cleansing composition of claim 2, wherein the cationic surfactant is cocamidopropyl-ammonium chloride.

5. The cleansing composition of claim 1, wherein the anionic surfactant is a carboxylate.

6. The cleansing composition of claim 1, wherein the water content of the composition is at least about 95%.

7. The cleansing composition of claim 1, further comprising a preservative.

8. The cleansing composition of claim 7, wherein the preservative is present in an amount of less than 1%.

9. The cleansing composition of claim 1, wherein the cleaning agent is present in an amount of less than 2%.

10. A cleansing composition for the cleaning of a mucosal membrane consisting essentially of:
at least one cleaning agent comprising a complex of an anionic surfactant and a non-anionic surfactant;
at least about 93% water; and
wherein the composition has an eye irritancy BCOP score of less than 1.

11. The cleansing composition of claim 10, wherein the non-anionic surfactant is a cationic surfactant.

12. The cleansing composition of claim 11, wherein the cationic surfactant is a polymeric cationic surfactant.

13. The cleansing composition of claim 11, wherein the cationic surfactant is cocamidopropyl-ammonium chloride.

14. The cleansing composition of claim 10, wherein the anionic surfactant is a carboxylate.

15. The cleansing composition of claim 10, wherein the water content of the composition is at least about 95%.

16. The cleansing composition of claim 10, further comprising a preservative.

17. The cleansing composition of claim 16, wherein the preservative is present in an amount of less than 1%.

18. The cleansing composition of claim 10, wherein the cleaning agent is present in an amount of less than 2%.

19. As cleaning system comprising:
a tissue substrate;
a cleansing composition having at least one cleaning agent comprising a complex of an anionic surfactant and a non-anionic surfactant, at least about 93% water; and wherein the composition has an eye irritancy BCOP score of less than 1; and
a dispenser for applying the cleansing composition to the tissue substrate.

20. The cleaning system of claim 19, wherein the dispenser is a touchless dispenser.

21. The cleaning system of claim 19, wherein the cleansing composition is contained in cannister.

22. The cleaning system of claim 19, wherein the cleansing composition comprises a complex of an anionic surfactant and a cationic surfactant.

23. The cleaning system of claim 19, wherein the cleansing composition comprises at least about 95% water, a preservative content of less than about 2% and a cleaning agent content of less than 2%.

24. A method of cleaning a mucosal membrane comprising:
applying a cleansing composition having at least one cleaning agent comprising a complex of an anionic surfactant and a non-anionic surfactant, at least about 93% water; and wherein the composition has an eye irritancy BCOP score of less than 1, to a tissue substrate to moisten the tissue substrate; and
contacting the mucosal membrane with the moistened tissue substrate to clean the mucosal membrane.

25. The method of claim 24, wherein the cleansing solution is applied from a touchless dispenser.

26. The method of claim 25, wherein the cleansing composition comprises a complex of an anionic surfactant and a cationic surfactant.

27. The method of claim 25, wherein the cleansing composition comprises at least about 95% water, and a preservative content of less than about 2%.

28. The method of claim 25, wherein the cleaning agent has a preservative content of less than 2%.
